(19) United States
(12) Patent Application Publication
(10) Pub. No.: US 2019/0083008 A1
(43) Pub. Date: Mar. 21, 2019

(54) UHF PHASED ARRAY RADAR FOR INTERNAL ORGAN DETECTION IN A MEDICAL SCANNER

(71) Applicant: Siemens Medical Solutions USA, Inc., Malvern, PA (US)

(72) Inventors: Ahmadreza Ghahremani, Knoxville, TN (US); James J. Hamill, Knoxville, TN (US)

(21) Appl. No.: 15/972,445

(22) Filed: May 7, 2018

Related U.S. Application Data
(60) Provisional application No. 62/560,698, filed on Sep. 20, 2017.

(51) Int. Cl. A61B 5/113; G01S 7/03; H01Q 3/34; A61B 5/05; A61B 5/00; A61B 5/11

(52) U.S. Cl. CPC A61B 5/1135; G01S 7/03; H01Q 3/34; G01S 13/89; A61B 5/7203; A61B 5/1126; A61B 5/6892; A61B 5/05

(57) ABSTRACT

A radar system for detecting displacement of an internal organ of a patient in a medical scanner. The system includes a plurality of antennas arranged relative to an axis of the patient, wherein at least one antenna is configurable as a transmitting antenna and at least two antennas are configurable as receiving antennas. The at least one antenna is also configurable as a receiving antenna and the at least two antennas are also configurable as transmitting antennas. The system also includes a phased array radar system that energizes the antennas to form a radiation pattern that is directed toward internal organ movement of the patient wherein the phased array radar system operates in the ultra high frequency (UHF) bandwidth of the electromagnetic spectrum.

### PRIORITY CLAIM

This application claims the benefit under 35 U.S.C. § 119(e) of copending U.S. Provisional Application No. 62/560,698 filed on Sep. 20, 2017 and entitled UHF PHASED ARRAY RADAR FOR INTERNAL ORGAN MOTION DETECTION IN A MEDICAL SCANNER, Attorney Docket No. 2017P08315US, which is incorporated herein by reference in its entirety and to which this application claims the benefit of priority.

### TECHNICAL FIELD

Aspects of the present invention relate to a radar system for detecting displacement of an internal organ of a patient positioned in a medical scanner, and more particularly, to a radar system having a plurality of antennas arranged relative to an axis of the patient, wherein at least one antenna is configurable as a transmitting antenna and at least two antennas are configurable as receiving antennas and wherein the at least one antenna is also configurable as a receiving antenna and the at least two antennas are also configurable as transmitting antennas, and wherein the system includes a phased array radar system that energizes the antennas to form a radiation pattern that detects internal organ movement of the patient wherein the phased array radar system operates in the ultra high frequency (UHF) bandwidth of the electromagnetic spectrum.

### BACKGROUND

Medical imaging techniques such as positron emission tomography (PET), computed tomography (CT), single-photon emission computed tomography (SPECT) and others are used to obtain images of the interior of a patient's body. During a diagnostic scan utilizing such imaging techniques, the patient's respiratory motion can cause undesirable image artifacts, or the incorrect alignment of two modalities due to internal organ movement that occurs during patient respiration.

In order to overcome these disadvantages, conventional imaging systems utilize respiration-correlated gating techniques to obtain a respiratory waveform. The waveform is then used to correlate respiration with time so as to provide motion correction of image data. Such systems typically include devices and sensors that are positioned on the patient by a trained operator. For example, a strain gauge or an optical tracker may be attached to a patient to measure chest elevation during respiration. However, the operation and accuracy of such systems is dependent on system setup and operator training. For example, pressure sensors used in some types of systems require adjustment by a trained operator prior to use. Further, the pressure sensors may loosen during a scan and require repositioning by the operator in order to maintain accuracy. In other types of systems that utilize optical detection of skin location, a line of sight path between a target and sensor is required that may be obscured by blankets, bent knees etc. of the patient. Moreover, the systems require substantial setup time and are not user friendly.

Alternatively, radar may be used to detect patient respiration. In one approach, continuous wave Doppler radar is used. However, such systems generate electromagnetic waves that reflect off surfaces located outside of the patient's body, such as a gantry surface of an imaging system, wall or other surface. This results in undesirable noise in the radar system and a relatively low signal to noise ratio (SNR) during operation of the CT gantry. Noise is also generated due to the application of common patch antennas having a uniform radiation pattern, use of a high gain directional patch antenna, placement of an antenna relatively close to the edge of a patient's bed and/or the patient's body position on the bed such as a patient's arms being raised up.

### SUMMARY OF THE INVENTION

A radar system for detecting internal organ displacement of a patient positioned in a medical scanner is disclosed. The system includes a plurality of antennas arranged relative to an axis of the patient, wherein at least one antenna is configurable as a transmitting antenna and at least two antennas are configurable as receiving antennas. In addition, the at least one antenna is also configurable as a receiving antenna and the at least two antennas are also configurable as transmitting antennas. The system also includes a phased array radar system that energizes the antennas to form a radiation pattern that detects internal organ movement of the patient wherein the phased array radar system operates in the ultra high frequency (UHF) bandwidth of the electromagnetic spectrum.

In addition, a method of operating a radar system for detecting internal organ displacement of a patient in a medical scanner is disclosed. The method includes providing a plurality of antennas arranged on a patient bed, wherein at least one antenna is configurable as a transmitting antenna and at least two antennas are configurable as receiving antennas and wherein the at least one antenna is also configurable as a receiving antenna and the at least two antennas are also configurable as transmitting antennas. The method also includes providing a phased array radar system that energizes the antennas to steer the main lobe of the radiation pattern toward diaphragm that detects internal organ movement of the patient in the medical scanner. In addition, the method includes transmitting radar signals in a direction of an examination region of a patient to detect internal organ movement of the patient. The extracted waveform based on the radar can be used to reduce motion blur in images using well-known techniques such as gating.

Those skilled in the art may apply the respective features of the present invention jointly or severally in any combination or sub-combination.

### BRIEF DESCRIPTION OF THE DRAWINGS

The exemplary embodiments of the invention are further described in the following detailed description in conjunction with the accompanying drawings, in which:

FIG. 1 depicts a plurality of patch antennas arranged in accordance with the invention.

FIG. 2 depicts the antennas located within a patient bed.

FIG. 3 shows a simplified block diagram of a radar system in accordance with the invention.

FIG. 4 depicts antennas arranged in a square array pattern in accordance with an aspect of the invention.

FIG. 5 shows an embodiment of a computed tomography (CT) system that includes the radar system.

FIGS. 6A and 6B are graphs depicting radar signals indicative of patient respiration detected before and after implementation of the invention, respectively, in two different modes wherein a CT gantry is not rotating (indicated by “static”) and wherein the CT gantry rotates (indicated by “rotating”).

To facilitate understanding, identical reference numerals have been used, where possible, to designate identical elements that are common to the figures. The figures are not drawn to scale.

### DETAILED DESCRIPTION

Although various embodiments that incorporate the teachings of the present disclosure have been shown and described in detail herein, those skilled in the art can readily devise many other varied embodiments that still incorporate these teachings. The scope of the disclosure is not limited in its application to the exemplary embodiment details of construction and the arrangement of components set forth in the description or illustrated in the drawings. The disclosure encompasses other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass direct and indirect mountings, connections, supports, and couplings. Further, “connected” and “coupled” are not restricted to physical or mechanical connections or couplings.

The entire disclosure of U.S. Patent Publication No. 2015/0002331 A1, published Jan. 1, 2015, entitled RADAR SYSTEM FOR MEDICAL USE by Allmendinger et al., is hereby incorporated by reference in its entirety.

In accordance with an aspect of the invention, phased array radar operating in the ultra high frequency (UHF) bandwidth of the electromagnetic spectrum (i.e. approximately 868 MHz - 930 MHz frequency range) is used to detect internal organ movement of a patient in a medical scanner. In an embodiment, the main beam of the antenna is directed into the patient's body and toward the thoracic diaphragm. Movement of the diaphragm is indicative of patient respiration. In an aspect, the invention enables detection of movement of the diaphragm as the patient breathes to generate a waveform indicative of patient respiration. It is known that the human body acts as a good absorber of UHF radio waves. By directing the main beam into the body and toward the diaphragm, the path length of electromagnetic waves that propagate within the human body is increased. As a result, the main beam of radiation of antenna is focused toward the target (at a selected angle). It has been found by the inventors herein high quality electromagnetic waves are reflected from the diaphragm as the diaphragm moves. In addition, the amount of radio frequency (RF) power leakage outside and around human body is substantially reduced which in turn substantially reduces the amount of undesirable reflections that occur from outside surfaces, thus substantially increasing a signal to noise ratio (SNR). Further, conventional systems provide coverage of a relatively small region of the patient. As a result, the patient is frequently relocated on the patient bed in order to obtain a suitable signal when using a conventional system. The excitation of a plurality of antennas, in accordance with the invention, provides coverage of larger region of the patient than that provided by conventional systems. Thus, the sensitivity of the radar can be decreased relative to a patient's position on a bed because an array of antennas have been excited. Since most part of the radio waves are absorbed inside human body, the invention is less sensitive relative to any movements near the bed like rotating CT gantry or bed movement during PET/CT scan.

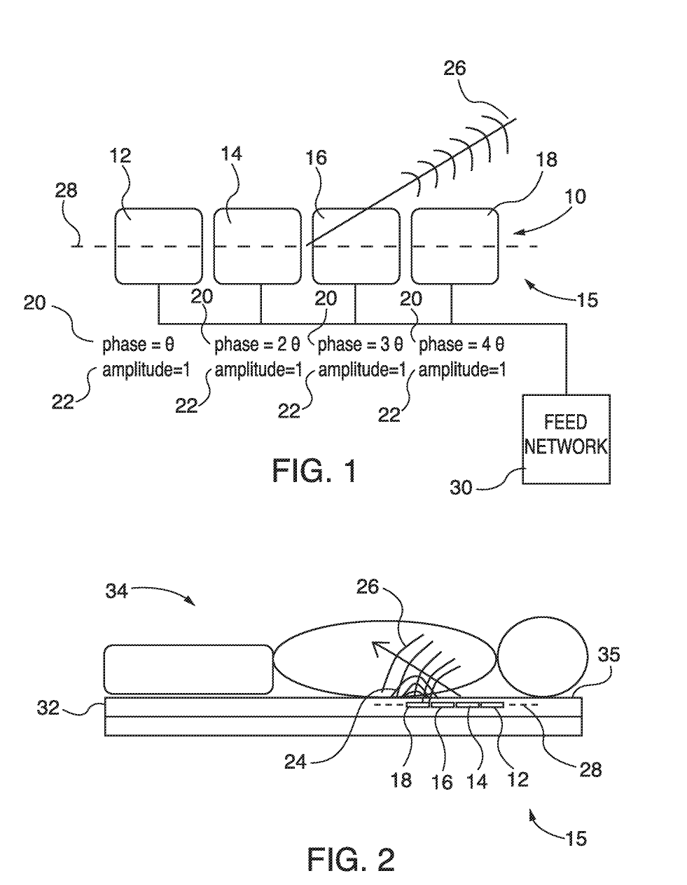

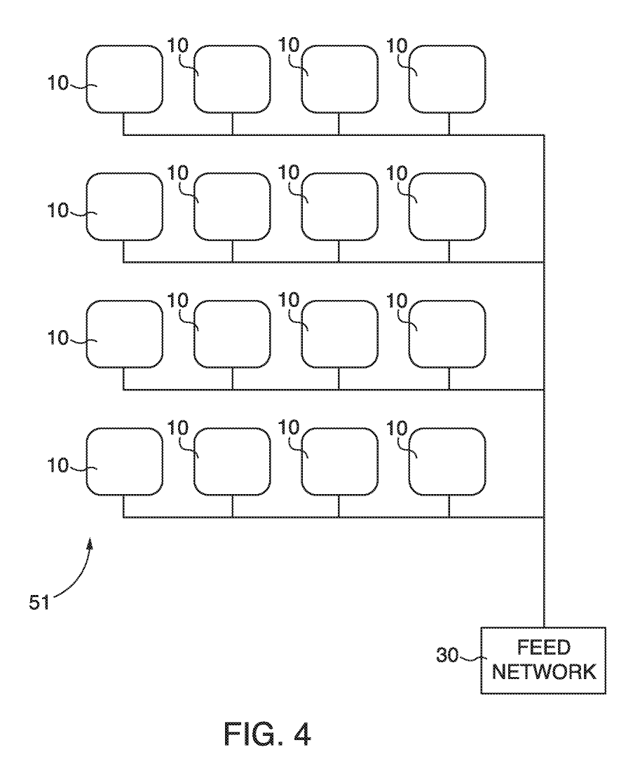

Referring to FIG. 1, a plurality of patch antennas 10 in accordance with the invention is shown. For purposes of illustration, FIG. 1 depicts first 12, second 14, third 16 and fourth 18 antennas although it is understood that additional or fewer antennas may be used. Each antenna 12, 14, 16, 18 generates radio waves that are electronically steered by a computer to form a phased array antenna arrangement 15. A phase angle θ 20 of an input signal used to excite consecutive antennas 12, 14, 16, 18 is increased while the amplitude 22 of the input signal for each antenna 12, 14, 16, 18 is maintained substantially similar to each other (i.e. the amplitude 22 of the input power is approximately 1 for each antenna 12, 14, 16, 18). In an embodiment, the phase angle θ 20 of the input signal for the antennas 12, 14, 16, 18 is θ, 2θ, 3θ, 4θ, respectively. It has been determined by the inventors herein that radio waves emitted by the antennas 12, 14, 16, 18 will achieve substantially far field electromagnetic radiation characteristics when they propagate within the human body in the vicinity of the diaphragm 24, such that the radio waves emitted by the antennas 12, 14, 16, 18 form a plane wave 26. Each antenna 12, 14, 16, 18 is fabricated from a material having a relatively high dielectric constant in order to reduce the size of each antenna 12, 14, 16, 18 at the UHF operating frequency. The antennas 12, 14, 16, 18 have a similar shape relative to each other. In an embodiment of the invention, the antennas 12, 14, 16, 18 are arranged along an antenna axis 28 to form a substantially in-line or linear arrangement that can operate at the same frequency. In accordance with aspects of the invention, the plurality of antennas 10 may be also arranged in other configurations suitable for steering the main lobe toward the internal organ being detected and/or obtaining a reflected signal as will be described in relation to FIG. 4. The plane wave 26 or main lobe of the radiation pattern is steered or directed by a feed network 30 that generates a fixed phase delay with respect to each antenna 12, 14, 16, 18. Known physical or electrical techniques may be employed in conjunction with the feed network 30 in order to generate the phase delay. For example, transmission lines such as those associated with power splitters may be configured to provide a physical delay. Alternatively, active microwave devices such as phase shifters at operation frequency may be used.

Referring to FIG. 2, the antennas 12, 14, 16, 18 are shown located within a patient bed 32. Alternatively, the antennas 12, 14, 16, 18 may be located on a surface 35 of the bed 32 or otherwise integrated into the bed 32. In another embodiment, the antennas 12, 14, 16, 18 may be located within or on a flexible mat 37 (see FIG. 4) placed on or under a patient 34. The invention enables a reduction in size of the antennas 12, 14, 16, 18 sufficient to enable location of the antennas 12, 14, 16, 18 under the patient 34. In an embodiment, the antenna axis 28 is aligned with a longitudinal axis of the patient (i.e. an axis of the patient extending in a direction between inferior and superior parts of the patient). For example, the antenna axis 28 may be aligned with the patient's backbone. Alternatively, the antenna axis 18 may be aligned with other axes of the patient 34 suitable for steering the main lobe toward the internal organ being detected and/or obtaining a reflected signal. The selected antennas 12, 14, 16, 18 may be configured as either receivers or transmitters. In an embodiment, the first 12, second 14 and third 16 antennas are configured as phased array receiver antennas 12, 14, 16 and the fourth antenna 18 is configured as a transmitter antenna 18. The receiver 12, 14, 16 antennas are located closest to the organ being detected relative to the transmitter antenna 18 so as to reduce an amount of interference and/or cross talk between the transmitter antenna 18 and receiver antennas 12, 14, 16. Further, the receiver antennas 12, 14, 16 are positioned to receive a substantial portion of the signals reflected from an internal organ being detected. Alternatively, the configuration of antennas 12, 14, 16, 18 may be reversed as desired such that antennas 12, 14, 16 are configured as phased array transmitter antennas 12, 14, 16 and the fourth antenna 18 is configured as a receiver antenna 18. It is understood that at least two of the antennas 12, 14, 16, 18 are required to form a phased array arrangement.

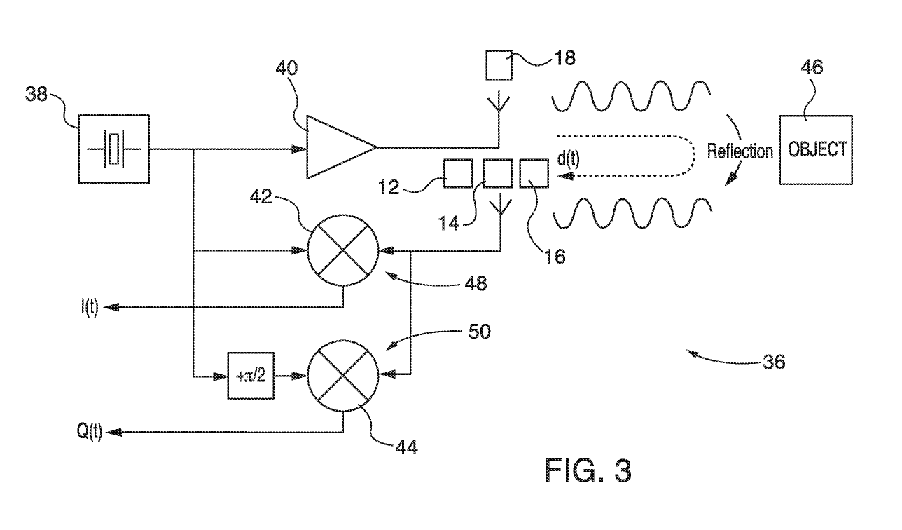

Referring to FIG. 3, a simplified block diagram of a radar system 36 in accordance with the invention is shown. The system 36 includes an oscillator 38, a power amplifier 40 and first 42 and second 44 I/Q mixers. After amplification by the power amplifier 40, a signal is emitted from the transmitter antenna 18 toward an object 46 such as a diaphragm of a patient. Radio waves reflected from the object 46 are then received by receiver 12, 14, 16 antennas. The resulting signal is mixed with the transmitted signal using first 42 and second 44 I/Q mixers. As the two signals have the same frequency, the mixing result is the phase difference between the signals. The magnitude of the output signals is the magnitude of the received signal minus a mixer conversion loss. The system 36 has two output channels denoted as I(t) and Q(t), the signals of which correspond to:

I(t)=Vᵢ+A cos(φ(t)+φ₀) Eqn. (1)

Q(t)=V_{q}+A sin(φ(t)+φ₀) Eqn. (2)

wherein I(t) is a reference signal, Q(t) is the signal shifted by 90 degrees, Vᵢ, V_{q}, and φ₀ denote constant offsets that are caused by parasitic effects such as antenna crosstalk or nonlinear behavior of the mixers 42, 44, A denotes the amplitude of the signal and φ(t) is the phase shift between transmitted and received signals. The phase shift φ(t) is proportional to the distance d(t) from the transmitting antenna to a reflection point on the object 46 and back to the receiver 12, 14, 16 antennas. A receiving unit have first 48 and second 50 channels is used in the system 36 to be still able to measure motion if one channel is in a so-called null point. This occurs if the mean distance between the object 46 and the antennas 12, 14, 16, 18 results in a phase shift near to an even multiple of it/2, where small changes of d(t) yield to I(t)=Vi=constant. To overcome this circumstance, the second mixer 44 of the second channel 50 receives an input signal from the oscillator 38 that includes a phase shift of π/2, so that its output is a sine function, as set forth in Eqn. (2). Thus, if one channel is in a null point, the other channel will be in an optimum point. The use of two channels 48, 50 is important since the distance between the antennas 12, 14, 16, 18 and the reflecting object 46 typically varies from patient to patient. An additional advantage of using two channels is that one of the channels can potentially determine the direction of motion.

In accordance with aspects of the invention, the plurality of antennas 10 may be arranged in any configuration suitable for steering the main lobe 26 toward the internal organ being detected and/or obtaining a reflected signal from the internal organ. Referring to FIG. 4, the antennas 10 may be arranged in a 4×4 square array 51, for example. It is understood that smaller or larger arrays and other array configurations may be used. For example, the antennas 10 may be arranged in a 2×4 array or other size array. In addition, the antennas 10 may be located relative to the patient 34 in any position suitable for steering the main lobe 26 toward the internal organ being detected and/or obtaining a reflected signal from the internal organ. For example, the mat 37, which may include an array of antennas 10, may be positioned either underneath, on top or on a side of the patient 34 as desired.

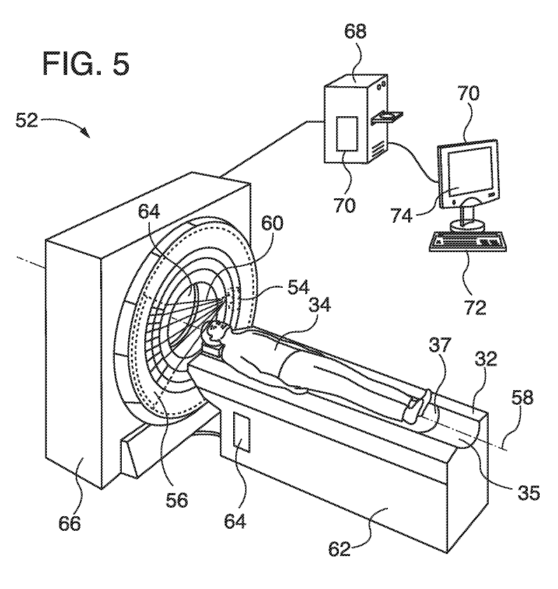

The invention may be used in conjunction with several types of medical imaging systems such as positron emission tomography (PET), single-photon emission computed tomography (SPECT), computed tomography (CT), PET/CT systems or radiation therapy systems. For purposes of illustration, the invention will be described in conjunction with a CT system 52 as shown in FIG. 5. The CT system 52 includes a recording unit, comprising an X-ray source 54 and an X-ray detector 56. The recording unit rotates about a longitudinal axis 58 during the recording of a tomographic image, and the X-ray source 54 emits X-rays 60 during a spiral recording. While an image is being recorded the patient 34 lies on the bed 32. The bed 32 is connected to a table base 62 such that it supports the bed 32 bearing the patient 34. The bed 32 is designed to move the patient 34 along a recording direction through an opening 64 of a CT gantry 66 of the CT system 52. In the example shown here the antenna arrangement 15 of the inventive radar system 36 is integrated into the bed 32.

The table base 62 includes a control unit 64 connected to a computer 68 to exchange data. The control unit 64 can actuate the system 36 (FIG. 3) and antenna arrangement 15 (FIGS. 1 and 2). In the example shown here the medical diagnostic or therapeutic unit is designed in the form of a CT system 52 by a determination unit 70 in the form of a stored computer program that can be executed on the computer 68. The computer 68 is connected to an output unit 70 and an input unit 72. The output unit 70 is for example one (or more) LCD, plasma or OLED screen(s). An output 74 on the output unit 70 comprises for example a graphical user interface for actuating the individual units of the CT system 52 and the control 64. Furthermore, different views of the recorded data can be displayed on the output unit 70. The input unit 72 is for example a keyboard, mouse, touch screen or a microphone for speech input.

### Test Results

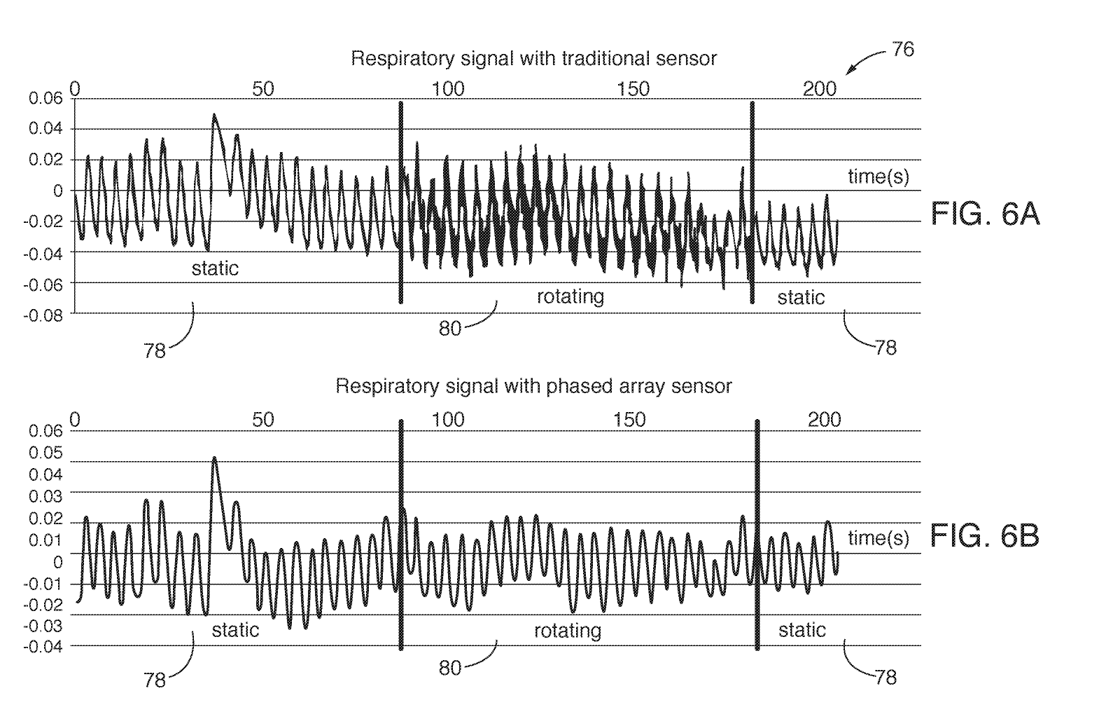

A test was conducted to detect radar signals from conventional patch antennas (i.e. not a phased array antenna in accordance with the invention) indicative of patient respiration during operation of a gantry of a PET/CT imaging system 52. During the test, a CT imaging function of the CT system 52 was not turned on (i.e. no X-rays were generated during the test). Referring to FIG. 6A, a graph 76 depicting radar signals indicative of patient respiration detected during operation of a CT gantry 66 is shown. A first region 78 of the graph 76 labeled as “static” indicates that the CT gantry 66 is not rotating during radar detection and thus there is no movement around the bed 32. A second region 80 of the graph 76 labeled “rotating” indicates that the CT gantry 66 is rotating 360 degrees around the bed 32 during radar detection. Any large peaks in the graph 76 are indicative of deep breathing. The measured data from radar detection illustrates that the radar signals are substantially affected by rotation of the CT gantry 66 (see the second region 80). To achieve the real signal, post-processing is required to filter the noise during rotation of CT gantry 66. When the test is repeated (i.e. a second test) while using a phased array antenna of the invention, the output radar signal for both the first 78 and second 80 regions will be clean and without any noise (see FIG. 6B). The second test (while using phased array antenna of the invention) indicates that noise in the system 36 is substantially reduced, thus reducing the need for complex filters and substantially improving the SNR of the radar's I and Q channels at the base band. Thus, the use of a phased array antenna arrangement 15 results in improved signal to noise in the radar's I and Q channels at the base band, in comparison with conventional radar used for respiratory gating system in any type of medical scanner.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

We claim:

1. A radar system for detecting displacement of an internal organ of a patient in a medical scanner, comprising:
a plurality of antennas arranged relative to an axis of the patient, wherein at least one antenna is configurable as a transmitting antenna and at least two antennas are configurable as receiving antennas and wherein the at least one antenna is also configurable as a receiving antenna and the at least two antennas are also configurable as transmitting antennas; and
a phased array radar system that energizes the antennas to form a radiation pattern that is directed toward internal organ movement of the patient.

2. The system according to claim 1, wherein the antennas are arranged within a patient bed that supports the patient.

3. The system according to claim 1, wherein the antennas are arranged within or on a mat located on or under the patient.

4. The system according to claim 1, wherein the phased array radar system operates in the ultra high frequency (UHF) bandwidth of the electromagnetic spectrum.

5. The system according to claim 1, wherein the antennas generate radio waves that achieve substantially far field electromagnetic radiation characteristics when they propagate within the patient's body near the organ.

6. The system according to claim 1, wherein the at least two receiving antennas are located closest to the organ being detected relative to the at least one transmitting antenna to reduce an amount of interference and/or cross talk between the transmitting and receiving antennas.

7. The system according to claim 1, wherein the organ is the thoracic diaphragm.

8. The system according to claim 1, wherein the axis is a longitudinal axis of the patient.

9. The system according to claim 1, wherein the antennas are arranged along an antenna axis.

10. The system according to claim 1, wherein the antennas are positioned to form an array.

11. A radar system for detecting displacement of an internal organ of a patient in a medical scanner, comprising:
a plurality of antennas arranged within or on a patient bed, wherein at least one antenna is configurable as a transmitting antenna and at least two antennas are configurable as receiving antennas and wherein the at least one antenna is also configurable as a receiving antenna and the at least two antennas are also configurable as transmitting antennas; and
a phased array radar system that energizes the antennas to form a radiation pattern that is directed toward internal organ movement of the patient wherein the phased array radar system operates in the ultra high frequency (UHF) bandwidth of the electromagnetic spectrum.

12. The system according to claim 11, wherein the antennas are arranged within or on a mat located on or under the patient.

13. The system according to claim 11, wherein the antennas generate radio waves that achieve substantially far field electromagnetic radiation characteristics when they propagate within the patient's body near the organ.

14. The system according to claim 11, wherein the at least two receiving antennas are located closest to the organ being detected relative to the at least one transmitting antenna to reduce an amount of interference and/or cross talk between the transmitting and receiving antennas.

15. The system according to claim 11, wherein a phase angle θ of an input signal used to excite four consecutive antennas is θ, 2θ, 3θ, 4θ, respectively.

16. The system according to claim 11, wherein the plurality of antennas includes first, second and third receiving antennas.

17. The system according to claim 11, wherein the organ is the thoracic diaphragm.

18. A method of operating a radar system to scan a patient positioned in a medical scanner, comprising:
providing a plurality of antennas arranged on a patient bed, wherein at least one antenna is configurable as a transmitting antenna and at least two antennas are configurable as receiving antennas and wherein the at least one antenna is also configurable as a receiving antenna and the at least two antennas are also configurable as transmitting antennas;
providing a phased array radar system that energizes the antennas to form a radiation pattern that detects internal organ movement of the patient;
transmitting radar signals in a direction of an examination region of a patient to detect internal organ movement of the patient.

19. The method according to claim 18, wherein the antennas are arranged within or on a mat located on or under the patient.

20. The method according to claim 18, wherein the antennas generate radio waves that achieve substantially far field electromagnetic radiation characteristics when they propagate within the patient's body near the organ.

21. The method according to claim 18, wherein the at least two receiving antennas are located closest to the organ being detected relative to the at least one transmitting antenna to reduce an amount of interference and/or cross talk between the transmitting and receiving antennas.

22. The method according to claim 18, wherein a phase angle θ of an input signal used to excite four consecutive antennas is θ, 2θ, 3θ, 4θ, respectively.

23. The method according to claim 18, wherein the plurality of antennas includes first, second and third receiving antennas.
